(19) United States
(12) Patent Application Publication
(10) Pub. No.: US 2008/0112894 A1
(43) Pub. Date: May 15, 2008

(54) Biocompatible Polymer Compounds For Medicinal Formulations

(76) Inventors: John T. Capecchi, Oakdale, MN (US); James S. Stefely, Woodbury, MN (US)

Correspondence Address: 3M INNOVATIVE PROPERTIES COMPANY, PO BOX 33427, ST. PAUL, MN, 55133-3427, US

(21) Appl. No.: 11/816,883

(22) PCT Filed: Mar. 14, 2006
(86) PCT No.: PCT/US06/08869

§ 371 Date: Aug. 22, 2007

Related U.S. Application Data
(60) Provisional application No. 60/661,370, filed on Mar. 14, 2005.

(51) Int. Cl. A61K 9/12; A61K 47/32

(52) U.S. Cl. 424/45; 514/772.6

(57) ABSTRACT

Stability of biocompatible polymers of the formula:

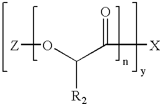

wherein Z is —C(O)R₁; each R₁ is independently selected from a linear, branched, or cyclic alkyl, alkoxy, or aryl with 1 to 18 carbon atoms optionally substituted by carbonyl, oxy, thio, and/or nitrogen; each R₂ is independently selected from hydrogen or a linear or branched hydrocarbon with 1 to 4 carbon atoms; X is selected from the group consisting of —OR₁, —SR₁, —N(R₁)₂ and divalent or trivalent headgroups terminated in oxygen, nitrogen, or sulfur; y is greater than or equal to 1 and less than or equal to 3; is enhanced by providing compositions where there is a low level of —OH end group impurity, i.e., where Z is —H instead of —C(O)R₁ and/or X is —OH instead of —OR₁, —SR₁, —N(R₁)₂ or divalent or trivalent headgroups terminated in —O—, —N—, or —S—, such that there are no more than 10 of these —OH end groups for every 100 intended biocompatible polymer molecules.

### CROSS-REFERENCE TO RELATED CASES

This application claims benefit of priority to U.S. provisional application 60/661,370, filed Mar. 14, 2005, the entire contents of which are hereby incorporated by reference.

### FIELD

The present invention relates to the use of biocompatible polymer compounds for medicinal formulations.

### BACKGROUND OF THE INVENTION

Biodegradable polymers have long been examined for their use in providing sustained release of drugs and have also been used to make biodegradable medical products. For example, polymeric esters of selected hydroxycarboxylic acids or their derivatives (e.g., lactic acid, glycolic acid, p-dioxanone, etc.) are known to be highly biocompatible with, and biodegradable in, the human body. Such polymers are degraded into their constituent hydroxycarboxylic acids, which are metabolized and eliminated from the body, over periods typically ranging from several weeks to several years. Consequently, compounds of this type have been utilized for such things as degradable sutures, preformed implants, and sustained release matrices.

Owing to this biocompatibility, polymers such as those derived from lactic acid (OLAs) and its derivatives have been used as excipients for metered dose inhalers (MDIs) and other medicinal applications. See, for example, U.S. Pat. No. 5,569,450 and U.S. Pat. No. 6,416,742. It is thus desirable to provide medicinal compositions with stable biocompatible polymer formulations, as well as methods for the manufacture of such biocompatible polymers and medicinal compositions.

### SUMMARY OF THE INVENTION

It has now been found that many biocompatible polymers, such as oligolactic acids, can suffer from impaired stability in medicinal formulations, such as MDIs. This is perhaps owing to the fact that such compounds are designed to degrade under physiological conditions. However, it has also been found that compositions of biocompatible polymer according to the formulas described below are made significantly more stable by reducing the amount of residual biocompatible polymer impurity having carboxylic acid and/or hydroxyl end groups.

The present invention can thus provide biocompatible polymers, methods, processes, compositions, and/or medicinal formulations that have desirable stability characteristics and are useful for drug delivery. They can be useful for, among other things, drug solubilization, chemical stabilization, and dispersing drug particle suspensions, as well as for providing sustained release of a drug via a drug delivery system, which can include, e.g., topical, implantable, and inhalation systems.

Many of the biocompatible polymers, methods, processes, compositions, and/or medicinal formulations are particularly useful for nasal and/or oral inhalation drug delivery, such as by inhalation from a metered dose inhaler. They can also be used as stabilizing or carrier matrices in dry powder inhaler (DPI) formulations and as suspension aids in hydrofluoroalkane (HFA) formulations.

In one embodiment, the present invention provides a medicinal formulation comprising: drug; biocompatible polymer of the formula:

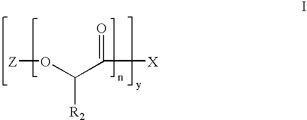

wherein Z is —C(O)R₁;
each R₁ is independently selected from a linear, branched, or cyclic alkyl, alkoxy, or aryl with 1 to 18 carbon atoms optionally substituted by carbonyl, oxy, thio, and/or nitrogen;
each R₂ is independently selected from hydrogen or a linear or branched hydrocarbon with 1 to 4 carbon atoms;
X is selected from the group consisting of —OR₁, —SR₁, —N(R₁)₂ and divalent or trivalent headgroups terminated in oxygen, nitrogen, or sulfur; y is greater than or equal to 1 and less than or equal to 3; and
wherein there is a low level of —OH end group impurity, where Z is —H and/or X is —OH, such that there are no more than 10 of these —OH end groups for every 100 biocompatible polymer molecules.

In one aspect of the aforementioned embodiment, R₁ is alkyl.

In one aspect of the aforementioned embodiments, R₁ is methyl.

In one aspect of the aforementioned embodiments, y is 2.

In one aspect of the aforementioned embodiment where y is 2, X is —NHCH₂CH₂NH—.

In one aspect of the aforementioned embodiments, there is a low level of —OH end group impurity, where Z is —H and/or X is —OH, such that there are no more than 1 of these —OH end groups for every 100 biocompatible polymer molecules.

In one aspect of the aforementioned embodiments, there is a low level of —OH end group impurity, where Z is —H such that there are no more than 1 of these —OH end groups for every 100 biocompatible polymer molecules.

In one aspect of the aforementioned embodiments, there is a low level of end group impurity, where X is —OH such that there are no more than 1 of these —OH end groups for every 100 biocompatible polymer molecules.

This contrasts with previous biocompatible polymer compositions of this type having considerably more than 10 of these —OH end group impurities to every 100 biocompatible polymer molecules.

In one embodiment, the present invention relates to the use of specific members of the family of relatively low molecular weight biocompatible polymeric compounds, broadly described as oligolactic acid and its derivatives (OLAs). These specific OLAs often have controlled polydispersities and controlled impurity levels. OLAs are often not a single chemical entity, but are in fact a collection of oligomers with a wide range of number average molecular weights. Specific OLAs, described as N,N′-ethylenebis(acetyloligolactyl)amides can provide particularly beneficial properties. The biocompatible polymers disclosed herein can allow for a broad spectrum of applications, including use as diluents and matrix forming compound powders (e.g., in DPIs, injectables) or as suspension aids in 1,1,1,2-tetrafluoroethane (also referred to as propellant 134a, HFC-134a, or HFA-134a) and in 1,1,1,2,3,3,3-heptafluoropropane (also referred to as propellant 227, HFC-227, or HFA-227) delivery systems. Surprisingly, these N,N′-ethylenebis(acetyloligolactyl) amides with low impurity levels exhibit superior stability over previously disclosed oligolactic acid compositions. This stability is observed in both the solid state and in HFA formulations. These OLAs can be stable when exposed to ambient conditions over extended periods of time. This increased stability enables their use in applications that may not have been desirable for other OLAs. In addition, the use of N,N′-ethylenebis(acetyloligolactyl)amides in medicinal formulations can be quite useful in MDI applications.

Although not intending to be bound to any particular theory or mechanism, it is hypothesized that one source of stability is the improved degree of acetylation of the hydroxy groups of the composition and the degree of derivatization of the acid functionality with the ethylenediamine core. It is hypothesized that full functionalization of the oligomer end groups minimizes the possibility of hydrolysis of the OLA ester linkages that could result in unstable formulations.

In one embodiment, the present invention provides a medicinal formulation, comprising:
drug;
biocompatible polymer N,N′-ethylenebis(acetyloligolactyl)amide of the formula:

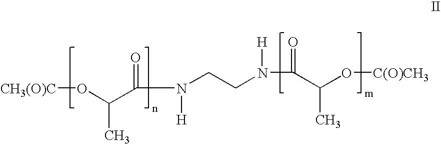

containing at least one unit derived from L-lactic acid and at least one unit derived from D-lactic acid;
wherein the average value of m and n is independently between 6 and 25; and
   wherein there is a low level of biocompatible polymer impurities having a hydroxy or carboxylic acid functional end group, such that there are no more than 5 of these —OH end groups for every 100 molecules of the biocompatible polymer N,N′-ethylenebis(acetyloligolactyl)amide.

In one embodiment, the average value of n is independently between 8 and 11.

In one embodiment, the average value of n is independently between 11 and 25.

In one embodiment, the medicinal formulation further comprises a propellant. The propellant may include 1,1,1,2-tetrafluoroethane and/or 1,1,1,2,3,3,3-heptafluoropropane.

In one embodiment, the biocompatible polymer comprises units derived from D,L-lactic acid.

In one embodiment, at least half of the stereocenters in the biocompatible polymer are derived from L-lactic acid.

In one embodiment, at least half of the stereocenters in the biocompatible polymer are derived from D-lactic acid.

In one embodiment, there is a low level of biocompatible polymer impurities having a hydroxy functional end group, such that there are no more than 1 of these —OH end groups for every 100 molecules of the biocompatible polymer N,N′-ethylenebis(acetyloligolactyl)amide.

In one embodiment, there is a low level of biocompatible polymer impurities having a carboxylic acid functional end group, such that there are no more than 1 of these —OH end groups for every 100 molecules of the biocompatible polymer N,N′-ethylenebis(acetyloligolactyl)amide.

In one embodiment, there is a low level of biocompatible polymer impurities having a hydroxy or carboxylic acid functional end group, such that there are no more than 1 of these —OH end groups for every 100 molecules of the biocompatible polymer N,N′-ethylenebis(acetyloligolactyl)amide.

In one embodiment, the polydispersity of the biocompatible polymer is less than or equal to 1.5.

In one embodiment, the number-average relative molecular mass of the biocompatible polymer is greater than 1200.

In one embodiment, the ratio of Mn/P of the biocompatible polymer is at least 900.

In one embodiment, the biocompatible polymer is synthesized by condensation polymerization.

In one embodiment, the medicinal formulation is free of metal-based catalysts.

In one embodiment, the drug is in solution.

In one embodiment, the drug is in suspension.

In one embodiment, the present invention provides a metered dose inhaler including a formulation as in any one of the aforementioned embodiments.

In one embodiment, the present invention provides a powder containing a formulation as in any one of the aforementioned embodiments.

In one embodiment, the present invention provides a dry powder inhaler including a formulation as in any one of the aforementioned embodiments.

In one embodiment, the present invention provides a method of stabilizing a medicinal formulation for use in a drug delivery system, the method comprising the steps of preparing a formulation as in any of the aforementioned embodiments and utilizing the formulation in a drug delivery system

In one embodiment, the present invention provides a method of treating in an animal a condition capable of being treated by a drug, the method comprising the steps of: (i) providing a formulation as in any of the aforementioned embodiments, and (ii) administering said formulation to said animal

In one embodiment, the present invention provides a medicinal formulation comprising: drug;
biocompatible polymer N,N′-ethylenebis(acetyloligolactyl)amide with an average number of repeat units in each oligolactic acid chain of between 8 and 11;
wherein the polydispersity of the N,N′-ethylenebis(acetyloligolactyl)amide is less than or equal to 1.5; and
wherein there is a low level of biocompatible polymer impurities having a hydroxy or carboxylic acid functional end group, such that there are no more than 5 of these —OH end groups for every 100 molecules of the biocompatible polymer N,N′-ethylenebis(acetyloligolactyl)amide.

As the terminology is used herein, a chain “derived from” a particular precursor need not be prepared from the precursor; rather this terminology is used to designate chains having a structure that could formally be obtained by condensation of the precursor.

Unless otherwise indicated, all numbers expressing quantities, ratios, and numerical properties of ingredients, reaction conditions, and so forth used in the specification and claims are to be understood as being modified in all instances by the term “about”.

All parts, percentages, ratios, etc. herein are by weight unless indicated otherwise.

As used herein, “a” or “an” or “the” are used interchangeably with “at least one” to mean “one or more” of the listed element. Also, the term “between” for a given range is intended to include the endpoints of the range (e.g., “between 8 and 11” would include both 8 and 11, as well as 9 and 10).

The term “lactic acid” when used without a “D”, “L”, or “DL” prefix refers to a molecule or mixture of molecules of lactic acid without specified stereochemistry.

The term “DL-lactic acid” refers to a racemic or 1:1 mixture of D-lactic acid and L-lactic acid.

The terms “polymer” and “polymeric” are, unless otherwise indicated, intended to broadly include homopolymers and block/random copolymers having a chain of at least three or more monomer structural units formed by polymerization reactions (e.g., condensation or ring-opening polymerization). The terms “oligomer” and “oligomeric” are used to refer to a subset of lower molecular weight polymers.

“Biocompatible polymer” refers generally to a polymer that is tolerated when placed within the body without causing significant adverse reactions (e.g., toxic or antigenic responses).

“Biodegradable polymer” refers to a polymer that degrades under biological conditions.

“Biological half-life” refers to the time required for half the mass of the material to disappear from the original site in vivo.

“Polydispersity” refers to the ratio of the weight-average to number-average molecular weights for a particular oligomeric or polymeric compound.

“Mn/P” refers to the ratio of the number-average molecular weight for a particular oligomeric or polymeric compound to the polydispersity of said particular compound.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### DETAILED DESCRIPTION OF THE INVENTION

The present invention provides medicinal formulations containing a drug and a biocompatible polymer compound. They can be solids, semi-solids, or liquids. Preferred formulations are delivered by nasal and/or oral inhalation, although formulations can also be made for delivery via other routes, for example, topical spray-on administration (e.g., buccal, transdermal). Additionally, compositions (e.g., those made with low polydispersity and/or medicinal salt biocompatible polymers) capable of forming stable preformed solid objects, such as dry powders, microspheres, rods, pins, etc., can be made for delivery by injection, implantation or other suitable methods, as well as nasal and/or oral inhalation.

As discussed below, the medicinal formulations may be made with a variety of drugs, biocompatible polymers, propellants, cosolvents, and other ingredients. Among the benefits provided by the invention, the biocompatible polymers may have enhanced physical and biodegradation properties due to low polydispersity, function as a solubilizing and/or chemical stabilizing aid, provide sustained release, and/or act as a counter ion to form a medicinal salt.

### Drugs

Medicinal formulations according to the present invention contain a drug (including combinations of drugs) either dispersed or dissolved in the formulation in a therapeutically effective amount (i.e., an amount suitable for the desired condition, route, and mode of administration). As used herein, the term “drug,” includes its equivalents, “bioactive agent,” and “medicament” and is intended to have its broadest meaning as including substances intended for use in the diagnosis, cure, mitigation, treatment or prevention of disease, or to affect the structure or function of the body. Administration of the medicinal formulation can be given to any animal. The drugs can be neutral or ionic. Preferably, they are suitable for nasal and/or oral inhalation. Delivery to the respiratory tract and/or lung, in order to effect bronchodilation and to treat conditions such as asthma and chronic obstructive pulmonary disease, is preferably by oral inhalation. Alternatively, to treat conditions such as rhinitis or allergic rhinitis, delivery is preferably by nasal inhalation.

Suitable drugs include, for example, antiallergics, analgesics, bronchodilators, antihistamines, antiviral agents, antitussives, anginal preparations, antibiotics, anti-inflammatories, immunomodulators, 5-lipoxygenase inhibitors, leukotriene antagonists, phospholipase A₂ inhibitors, phosphodiesterase IV inhibitors, peptides, proteins, steroids, and vaccine preparations. A group of preferred drugs include adrenaline, albuterol, atropine, beclomethasone dipropionate, budesonide, butixocort propionate, clemastine, cromolyn, epinephrine, ephedrine, fentanyl, flunisolide, fluticasone, formoterol, ipratropium bromide, isoproterenol, lidocaine, morphine, nedocromil, pentamidine isoethionate, pirbuterol, prednisolone, salmeterol, terbutaline, tetracycline, 4-amino-α,α,2-trimethyl-1H-imidazo[4,5-c]quinoline-1-ethanol, 2,5-diethyl-10-oxo-1,2,4-triazolo[1,5-c]pyrimido[5,4-b][1,4]thiazine, 1-(1-ethylpropyl)-1-hydroxy-3-phenylurea, and pharmaceutically acceptable salts and solvates thereof, and mixtures thereof.

For nasal and/or oral inhalation, formulations where the drug is in solution and chemically stable are generally preferred; however, if suspensions are used, preferably the drug is micronized (i.e., in the form of particles having a diameter on the order of micrometers). In one aspect, a therapeutically effective fraction of the drug (typically, about 90% or more) is in the form of particles having a diameter of less than 500 micrometers, less than 50 micrometers, or less than about 5 micrometers. These particle sizes also apply for the formulations (drug and biocompatible polymer) used in dry powder inhalers. This ensures that the drug can be inhaled into the respiratory tract and/or lungs. It will be recognized that such limitations do not necessarily exist for nasal inhalation.

In one embodiment, medicinal formulations according to the present invention provide for a drug in an amount and in a form such that the drug can be administered as an aerosol. More preferably in such applications, the drug is present in an amount such that the drug can produce its desired therapeutic effect with one dose from a conventional aerosol canister with a conventional valve, such as a metered dose valve. As used herein, an “amount” of the drug can be referred to in terms of quantity or concentration. A therapeutically effective amount of a drug can vary according to a variety of factors, such as the potency of the particular drug, the route of administration of the formulation, the mode of administration of the formulation, and the mechanical system used to administer the formulation. A therapeutically effective amount of a particular drug can be selected by those of ordinary skill in the art with consideration of such factors. Generally, a therapeutically effective amount will be from about 0.02 parts to about 2 parts by weight based on 100 parts of the medicinal formulation for propellant containing formulations.

In one embodiment, medicinal formulations according to the present invention provides for a drug in an amount and in a form such that the drug can be administered as particulate medicinal formulation for use in a dry powder inhaler. Generally, a therapeutically effective amount will be from about 0.02 parts to about 99 parts by weight based on 100 parts of the dry powder medicinal formulation

### Biocompatible Polymers

In one embodiment, biocompatible polymers of the present invention have the following formula:

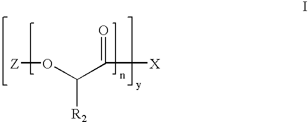

Each chain or chains in the biocompatible polymer is capped on one end by an end group, Z, wherein Z is —C(O)R₁. Each R₁ is independently selected from a linear, branched, or cyclic alkyl, alkoxy, or aryl with 1 to 18 carbon atoms optionally substituted by carbonyl, oxy, thio, or nitrogen. In one embodiment where y is greater than 1, the R₁ substituent on each chain is equivalent. Suitable examples of R₁ include unsubstituted alkyl, alkoxy, or aryl with 1 to 18 carbon atoms, preferably methyl or ethyl, and most preferably methyl.

The choice of end group may modify the performance of the polymer, either in the formulation or biologically. It is preferred for regulatory and biological reasons to minimize the complexity of the biocompatible polymer. However, for physical and chemical reasons it may be preferable to modify the biocompatible polymer with respect to increased stability, propellant solubility (e.g., in hydrofluorocarbons), water affinity/solubility, interaction with the drug, etc. Such parameters frequently influence drug release rates. Preferred biocompatible polymers as described herein contain at least one chain capped with an organocarbonyl group, and more preferably, with an acetyl group. Acylation can significantly enhance stability and reduce the hydrophilicity and water solubility of the biocompatible polymers.

Each R₂ is independently selected from hydrogen or a linear or branched hydrocarbon with 1 to 4 carbon atoms. Each R₂ in a chain of units may be identical, for example, each R₂ in a chain may be methyl (and thus derived from lactic acid). Alternatively, more than one distinct R₂ may be present in a chain, for example, a chain may contain a mixture of units where R₂ is methyl and hydrogen (and thus derived from a mixture of lactic acid and glycolic acid). A mixture of units may be present in each chain in a random sequence or an ordered sequence, such as in a block or alternating structure. In one embodiment where y is greater than 1, the distribution of R₂ substituents on each chain is equivalent. In one embodiment R₂ is methyl.

Each chain or chains in the biocompatible polymer is capped or bridged on one end by a headgroup, X. Suitable examples of the headgroup X are selected from the group consisting of —OR₁, —SR₁, —N(R₁)₂, and divalent or trivalent headgroups terminated in —O—, —N—, or —S.

A chain may be capped by a monovalent, divalent, or polyvalent organic moiety (each valence of the capping group being independently bonded to a chain) that does not contain hydrogen atoms capable of hydrogen bonding. The chain may also be capped at one end or both ends by a monovalent, divalent, or polyvalent group, selected from either an ionic group or a group that does contain hydrogen atoms capable of hydrogen bonding. Such groups need not necessarily terminate the compound; rather, they can bridge chains, which is the case when y is greater than 1. Where y is equal to 1 the chain is typically capped by a monovalent headgroup. Where y is greater than 1, two or more chains are bridged by a divalent or polyvalent headgroup. In one embodiment, y is greater than or equal to 1 and less than or equal to 3. In one embodiment, y is 2.

Examples of groups not containing hydrogen atoms capable of hydrogen bonding include organocarbonyl groups such as acetyl and alkoxy groups such as ethoxy. Examples of ionic groups include quaternary ammonium groups, sulfonate salts, carboxylate salts, and the like. Examples of groups capable of hydrogen bonding include hydrogen when bonded to a heteroatom terminus of a chain, as well as acid functional groups, amides, carbamates, and groups such as amino, hydroxyl, thiol, aminoalkyl, alkylamino, hydroxyalkyl, hydroxyalkylamino, sugar residues, and the like. Such end groups are well known and can be readily selected by those skilled in the art, and are disclosed, for example, in U.S. Pat. Nos. 5,569,450 and 6,042,811, the disclosures of which are herein incorporated by reference. In one embodiment, end groups may be selected which are poor nucleophiles and/or which lack sufficient acidity to catalyze degradation of the biocompatible compound.

Preferred biocompatible compounds comprise condensation-type homopolymers or block or random copolymers. In one embodiment, the compounds comprise N,N′-ethylenebis(acetyloligolactyl)amide of the formula

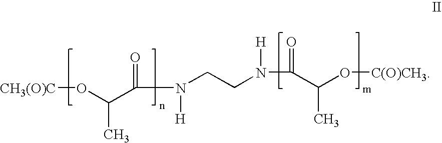

The chain of units within formulas (I or II) can be derived from a precursor hydroxyacid. As used herein, a chain “derived from” a particular precursor need not be prepared from the precursor; rather, this terminology is used to designate chains having a structure that could formally be obtained by condensation of the precursor. For example, the chain of units of formula I can be typically referred to as lactic acid condensate units, although these need not necessarily be prepared by the condensation of lactic acid. Rather, this terminology is used to designate chains having a structure that could in principle be obtained by a condensation reaction of lactic acid. A precursor hydroxyacid can be any hydroxyacid, such as a hydroxy carboxylic acid, or the corresponding lactone or cyclic carbonate, if any. Suitable lactones include L-lactides, D-lactides, or any combination thereof.

It is preferred that the precursor hydroxyacids be a mixture of L-lactic acid and D-lactic acid. In one embodiment, the precursor hydroxyacid is DL-lactic acid. The chain of units may contain any ratio of units derived from D-lactic acid and L-lactic acid ranging from 1000:1, 100:1, 10:1; 1:1, 1:10; 1:100, or 1:1000. The preferred ratio of units derived from D-lactic acid and L-lactic acid in the chain of units of formula I is 1:1. The DL form has some advantages due to its amorphous nature and enhanced solubility in, for example, hydrofluorocarbon propellants such as HFC 134a and 227. The L form is also advantageous as it is endogenous to the human body.

One skilled in the art can select units for inclusion in the chains of the biocompatible polymers with consideration of factors, such as mode of administration, ease of metabolism, solubility or dispersibility, crystallinity, structural homogeneity, molecular weight, other components to be used in the medicinal formulations, etc. A chain can be formally derived from any combination of L-lactic acid and D-lactic acid units. The chains can possess any sequence of units that can be derived from L-lactic acid and D-lactic acid. The sequence of the units derived from L- and D-isomers can be random or can have a contiguous sequence of a single isomer for a portion or entire chain length. The sequence may also possess a repeating structure comprising units derived from both L- and D-lactic acid units. In the embodiment where y is greater than 1, each sequence of units in the biocompatible compound may have a different isomeric composition.

Preferably, biocompatible polymer compounds described herein are also biodegradable. As used herein, a “biocompatible” polymer or compound is one that does not generally cause significant adverse reactions (e.g., toxic or antigenic responses) in the body, whether it degrades within the body, remains for extended periods of time, or is excreted whole. A “biodegradable” polymer or compound is one that relatively easily degrades under biological conditions. Typically, biodegradation occurs initially by way of hydrolytic degradation (i.e., hydrolysis of the polymers into smaller molecules).

Biocompatible compounds described herein can have a wide variety of molecular weights. Typically, they should have a number-average molecular weight of no greater than about 5000. Depending on the particular embodiment and purpose(s) of the biocompatible polymer used therein, the compounds described herein may have a number-average molecular weight of at least about 1000, at least about 1200, or at least 2000 for each chain of units. The biocompatible polymers will usually have a preferred chain length, or average value of n, of at least 6, at least 11, or at least 20 units. The average value of n is independently selected to be between 3 and 70, often between 6 and 25, and sometimes between 3 and 25. In one aspect, the average value of n is independently selected to be between 8 and 11. In another aspect, the average value of n is independently selected to be between 11 and 25. In one embodiment, the values of n are equal when y is greater than 1.

In some embodiments, it is preferable that the compound be substantially free of water-soluble polymers so that, for example, the polymer does not quickly dissolve upon delivery to the body tissue, such as the lung, but rather degrades over a desired time period. Generally, the polymers having less than 8 repeat units tend to be water soluble, while polymers having 8 or more repeat units tend to be relatively insoluble, although the precise chain length of course varies with the nature of the repeat units and the nature of the chain end capping units.

These various preferred molecular weights and chain lengths are by necessity only general guidelines since there are many factors, as will be understood by those skilled in the art, such as the particular polymer type, end-cap groups, and the presence and type of other ingredients (propellants, excipients, etc.), which can greatly affect the choice of molecular weight used.

It is well known that polymers contain a distribution of chain lengths. A particularly preferred embodiment of the present invention has a narrow range of chain lengths, thereby providing a biocompatible polymer having a relatively narrow molecular weight distribution, i.e., low polydispersity. One skilled in the art will recognize the particular distribution that is preferred for a given application based on the degree of solubility, bulk physical characteristics, biological compatibility and degradation, formulation processability, and performance factors (e.g., solubilizing ability, drug release rate control, shelf life, dose reproducibility, etc.) of the compound.

For certain embodiments of the present invention, suitable biocompatible polymers preferably have a relatively narrow molecular weight distribution. Generally, for such embodiments, the polydispersity (i.e., the ratio of weight-average to number-average molecular weight) is less than about 1.8 or less than about 1.5. This is particularly true for certain sustained release formulations utilizing higher molecular weight polymers. In some embodiments, the polydispersity is preferably less than about 1.4. Relatively narrow molecular weight distributions may be desirable to improve physical characteristics of the composition in solid form, to enhance solubility in, for example, an aerosol propellant, or to provide a material that has an optimized rate of biodegradation. In certain applications this results in an appropriate rate of drug release and improved shelf life and handling characteristics in its bulk form.

One skilled in the art will recognize that these parameters will vary with each monomer used. For example, when poly-DL-lactic acids of normal polydispersity are used in a formulation for pulmonary delivery, it is preferred that the number-average molecular weight of the polymer be no greater than about 1800. Otherwise, depending upon the frequency of administration, the higher molecular weight component present can accumulate in the lung. When narrow molecular weight range poly-DL-lactic acids (i.e., those having a polydispersity of less than about 1.15) are used, however, the preferred number-average molecular weight is preferably no greater than about 2000, and more preferably, for most applications, no greater than about 1600. In general, it is desirable to use the lowest molecular weight biocompatible polymer that still provides adequate incorporation of the drug into the polymer matrix upon delivery, along with the desired release rates.

In certain embodiments, it is desirable for the chain of units comprising the compound to be both sufficiently large (i.e., have a larger number-average molecular weight) and possess a relatively narrow molecular weight distribution (i.e., the polydispersity). A measurement of these desirable characteristics in certain embodiments can be expressed as a ratio of the number-average molecular weight to the polydispersity. In these embodiments, the ratio (Mn/P) is desired to be at least 900, at least 1200 or at least 2000. In some embodiments, the ratio (Mn/P) will be desired to be no greater than 2500. One skilled in the art will recognize which distribution is preferred for a given application based on the degree of solubility, bulk physical characteristics, biological compatibility and degradation, formulation processability, and performance factors (e.g., solubilizing ability, drug release rate control, shelf life, dose reproducibility, etc.) of the compound.

As already noted, it is generally preferred that the biocompatible polymers of the present invention are biodegradable. Typically, such polymers are sufficiently biodegradable such that they have a biological half-life (e.g., in the lung) of less than about 10 days, often less than about 4 days, sometimes less than about 2 days, and occasionally less than about 1 day. For certain embodiments of the present invention, biocompatible polymers are sufficiently biodegradable in use such that medicinal formulations containing them have a biological half-life of less than about 7 days. In one embodiment, such as those formulations capable of being inhaled, the biological half-life may be less than about 2 days (often less than about 1 day, sometimes less than about 12 hours, and occasionally less than about 6 hours). As used herein, “biological half-life” is the time required for half the mass of the material to disappear from the original site in vivo.

Thus, certain preferred compounds described herein can be combined with a drug to form a rapidly degrading, morphologically shelf stable polymeric matrix, which can be in the form of a dispersion, or dry powder, for example. Such biocompatible polymers are preferably homo-polymers having linear chains of units derived from an alpha-hydroxy carboxylic acid, such as DL-lactic acid, and preferably have a polydispersity of less than about 1.45.

The optimal amount of the biocompatible polymer depends on its nature, what role it serves within the formulation, and the nature of the drug with which it is used. A practical upper limit in aerosol formulations is based on the solubility of the polymer. The solubility levels of individual biocompatible polymers are a function of the molecular weight and polydispersity of the polymer, as well as the chemical nature of the repeating units and endgroups. In general, the solubilities of the polyhydroxycarboxylic acids (for a given molecular weight) increase as their tendency toward crystallization decreases. For example, as the ratio of D to L isomers approaches 1:1 for a chain, the solubility of a given compound will generally increase.

For propellant-based aerosol formulations, such as an MDI, the biocompatible polymer may be present in dissolved form in an amount of from about 0.01 part to about 25 parts by weight based on 100 parts of the medicinal formulation, preferably from about 0.1 part to about 10 parts by weight based on 100 parts of the medicinal formulation, and for some applications preferably from about 1 part to about 5 parts by weight based on 100 parts of the medicinal formulation.

In one embodiment, the biocompatible polymer may be present in a solid form, such as in a dry powder inhaler, in an amount of from about 0.2 parts to about 99.9 parts by weight based on 100 parts of the medicinal formulation, preferably from about 50 parts to about 98 parts by weight based on 100 parts of the medicinal formulation.

### Method to Produce Compounds

In one embodiment, the present invention provides a method of producing a polymeric compound (e.g., N,N′-ethylenebis(acetyloligolactyl)amides) having, for example, a number-average molecular weight greater than about 1200, and a significantly reduced polydispersity (e.g., less than about 1.8 and, preferably, less than about 1.5). The method comprises polymerization of the lactic acid via condensation followed by capping the hydroxyl end of the polymer with a capping group. This capping group is often an acetyl group. Ethylenediamine can then be coupled to the oligolactic acid via condensation and formation of the amide.

In one embodiment, the present invention provides a method of producing a polymeric compound of formula I. The method comprises polymerization of an alpha-hydroxy acid via condensation followed by capping the hydroxyl end of the polymer with a capping group. This capping group is often an acetyl group. The carboxyl group of the poly alpha-hydroxy acid is capped or bridged on one end by a headgroup, X, such as described above. For example, ethylenediamine can be coupled to the carboxyl functionality via condensation and provide formation of an amide.

These reactions can be run in solution, and the solvent can also serve as the propellant in the formulation, if applicable. Preferred solvents that can also serve as propellants include 1,1,1,2-tetrafluoroethane (also referred to as propellant 134a, HFC-134a, or HFA-134a) and/or 1,1,1,2,3,3,3-heptafluoropropane (also referred to as propellant 227, HFC-227, or HFA-227). Examples of suitable synthetic methods for polymerizing and capping polymers may be found in U.S. Patent Application Nos. 60/533,172 (“Medicinal Compositions and Method for the Preparation Thereof”, Capecchi et al.) and 60/613,063 (“Medicinal Aerosol Formulations and Methods of Synthesizing Ingredients Therefor”, Bechtold et al.), the disclosures of which are herein incorporated by reference.

The method of polymer condensation provides significant advantages. Besides the unexpected superiority of the products, it also provides advantages over other polymerizations that utilize metal-based catalysts, which are more expensive, present environmental disadvantages, and raise health concerns due to residual contamination.

The method may also provide improved degrees of acylation or acetylation of the OH endgroups and of the degree of derivatization of the acid functionality with a capping or bridging group, such as ethylenediamine. In one aspect, the method provides for degrees of completion such that the molar ratio of unreacted oligolactic acid and oligolactic acid derivatives having a free hydroxyl is less than 10%, less than 5%, or less than 1% of the amount of N,N′-ethylenebis(acetyloligolactyl)amide prepared. In one aspect, the method also provides for degrees of completion such that the molar ratio of unreacted oligolactic acid and oligolactic acid derivatives having a free carboxylic acid is also less than 10%, less than 5%, or less than 1% of the amount of N,N′-ethylenebis(acetyloligolactyl)amide prepared.

In one aspect, the method provides for degrees of completion that minimize the amount of biocompatible polymer impurities characterized by formula I with the exception that Z is —H and/or X is —OH. When X is —OH the impurity may also be described as having a carboxylic acid functionality. These impurities may be, for example, unreacted starting materials wherein Z is substituted by —H and/or X is —OH. The method may provide for degrees of completion such that the molar ratio of the amount of unreacted biocompatible polymer impurity to the amount of biocompatible polymer I prepared is less than or equal to 10%, often less than or equal to 5%, and sometimes less than or equal to 1%. The method may provide for degrees of completion such that the molar ratio of the amount of unreacted biocompatible polymer impurity wherein Z is substituted by —H to the amount of biocompatible polymer I is less than or equal to 10%, often less than or equal to 5%, and sometimes less than or equal to 1%. The method may provide for degrees of completion such that the molar ratio of the amount of unreacted biocompatible polymer impurity wherein X is —OH in the formulation to the amount of biocompatible polymer I is less than or equal to 10%, often less than or equal to 5%, and sometimes less than or equal to 1%. Determination of the relative amount of impurity may be determined by conventional analytical methods, such as, for example, nuclear magnetic resonance (NMR) or liquid chromatography-mass spectrometry (LC-MS).

### Medicinal Aerosol Formulations

One embodiment of the present invention provides a medicinal aerosol formulation including a propellant, a drug, and a soluble biocompatible polymer. Such medicinal formulations are preferably suitable for nasal and/or oral inhalation. By this it is meant, among other things, that when delivered from a metered dose inhaler they form particles of a size appropriate for nasal and/or oral inhalation and do not typically form films. These particles are formed spontaneously as the formulation exits the aerosol valve and the propellant evaporates. Hence, although the biocompatible polymers described herein may be used to make preformed sustained release microparticles (e.g., microspheres) by conventional means, the present invention also provides a method for automatically generating sustained release microparticles from an aerosol spontaneously upon valve actuation, without requiring any preformed microparticles. That is, the method includes the steps of: preparing a medicinal aerosol formulation by combining components comprising a propellant, and a biocompatible polymer substantially completely soluble in the medicinal formulation to provide for sustained drug release, and a drug as a micronized suspension or substantially completely dissolved in the medicinal formulation in a therapeutically effective amount; placing the medicinal formulation in a device capable of generating an aerosol (e.g., an aerosol canister equipped with a valve, and more preferably, an aerosol canister equipped with a metered dose valve); and actuating the device to form an aerosol comprising particles suitable for delivery to a body site, such as the lung or nasal passages.

In one embodiment, an aerosol formulation may be a sustained release formulation, that is, one that releases the drug over an extended period of time (e.g., as short as about 60 minutes or as long as several hours and even several days or months), rather than substantially instantaneously upon administration. Typically, for a polymer matrix of a particular size, the sustained release characteristics are determined by the nature of the biocompatible polymer and of the drug. Also, it is determined by the relative amount of biocompatible polymer to drug.

A sustained release medicinal formulation may include a biocompatible polymer in an amount such that the period of therapeutic activity of the drug is increased relative to the activity of a like formulation having propellant and drug but without the biocompatible polymer. Preferably, this increase is by a factor of at least about 1.5. Alternatively, for certain embodiments, the sustained release medicinal formulation may include a biocompatible polymer in an amount and type such that the period of therapeutic activity of the drug is extended by the presence of the biocompatible polymer by at least about 30 minutes, and often by at least about 2 hours, and sometimes by at least about 6 hours. When used in aerosol formulations, it will be understood by one of skill in the art that a direct comparison of a like formulation without the biocompatible polymer may not be meaningful due to formulation difficulties when the biocompatible polymer is absent. Thus, a conventional dispersant and/or cosolvent may need to be added to the medicinal formulation to provide an inhalable formulation for comparison of the period of time during which the drug is present at levels needed to obtain a desired biological response.

The amount of biocompatible polymer (total mass relative to drug) that will be sufficient to provide sustained release over a desired period of time depends, among other things, on the form of the drug. In the case of aerosol formulations containing the drug in micronized particle form (i.e., dispersed in the formulation), the amount of biocompatible polymer (preferably, biodegradable polymer) is generally enough to provide a substantially complete layer or coating around the micronized particles after exiting the aerosol valve. This amount is typically considerably greater than the amount that is used when such polymers are used solely as dispersing aids. It may be at least about a 1:1 molar ratio of biocompatible compound to drug. Sometimes, the molar ratio of biocompatible compound to drug is greater than about 4:1 on a molar basis. Alternatively, on a weight basis it may be at least about a 1:1 ratio of biocompatible polymer to drug. Often, on a weight basis there will be at least about a 4:1 ratio, and sometimes at least about an 8:1 ratio of biocompatible compound to drug.

In the case of aerosol formulations containing the drug in solution (i.e., substantially completely dissolved in the formulation), the amount of biocompatible polymer (preferably, biodegradable polymer) sufficient to provide sustained release varies considerably. In general, at least about a 1:1 molar ratio of biocompatible polymer to drug is desirable, although lesser amounts may be used to provide partial sustained release (e.g., bi-phasic release, etc.) and/or as a solubilization aid for the drug. Alternatively, on a weight to weight basis, the ratio of polymer to drug is generally between about 1:1 and about 100:1. Preferably, the amount of biocompatible polymer for sustained release of a drug in dissolved form is typically between about 2:1 to about 30:1 weight ratio of biocompatible polymer to drug, and more preferably, about 4:1 to about 15:1 on a weight basis. Again, however, the desired amount can depend on many factors, including the desired release times, nature of the drug or agents involved, the nature and number of biocompatible polymers used, as well as the average molecular weight(s) of the biocompatible polymer(s) and their polydispersities. In general, larger weight ratios of polymer to drug will lead to slower drug release rates. Those skilled in the art will be readily able, based on the teachings herein, to incorporate and assess the various factors to suit a particular application of the invention.

In one embodiment, formulations of the present invention may contain certain biocompatible polymer impurities related to the biocompatible polymers of formula I. These biocompatible polymer impurities can be characterized by formula I with the exception that Z is —H and/or X is —OH. These impurities may be related to or caused by impurities in starting materials used to synthesize the biocompatible polymers, related to or caused by incomplete derivatization reactions of the starting materials, and/or related to or caused by degradation of the formulation components during manufacturing or storage. An unmodified oligolactic acid is an example of such a biocompatible polymer impurity. In one embodiment, the molar ratio of the amount of biocompatible polymer impurity in the formulation to the amount of biocompatible polymer I is less than or equal to 10%, often less than or equal to 5%, and sometimes less than or equal to 1%. In one embodiment, the molar ratio of the amount of biocompatible polymer impurity wherein -Z is —H in the formulation to the amount of biocompatible polymer I is less than or equal to 10%, often less than or equal to 5%, and sometimes less than or equal to 1%. In one embodiment, the molar ratio of the amount of biocompatible polymer impurity wherein X is —OH in the formulation to the amount of biocompatible polymer I is less than or equal to 10%, often less than or equal to 5%, and sometimes less than or equal to 1%. In one embodiment, there is a low level of —OH end group impurity, where Z is —H and/or X is —OH, such that there are no more than 10, often nor more than 5, and sometimes no more than 1 of these —OH end groups for every 100 biocompatible polymer molecules. In one embodiment, there is a low level of biocompatible polymer impurities having a hydroxy or carboxylic acid functional end group, such that there is no more than 1 of these —OH end groups for every 100 molecules of the biocompatible polymer.

### Propellants

Medicinal formulations according to the present invention may include a propellant. Suitable propellants include, for example, a chlorofluorocarbon (CFC), such as trichlorofluoromethane (also referred to as propellant 11), dichlorodifluoromethane (also referred to as propellant 12), and 1,2-dichloro-1,1,2,2-tetrafluoroethane (also referred to as propellant 114), a hydrochlorofluorocarbon, a hydrofluorocarbon (HFC), such as 1,1,1,2-tetrafluoroethane (also referred to as propellant 134a, HFC-134a, or HFA-134a) and 1,1,1,2,3,3,3-heptafluoropropane (also referred to as propellant 227, HFC-227, or HFA-227), carbon dioxide, dimethyl ether, butane, propane, or mixtures thereof. Preferably, a hydrofluorocarbon is used as the propellant. More preferably, HFC-227, HFC-134a, and mixtures thereof are used as the propellant. The propellant is preferably present in an amount sufficient to propel a plurality of doses of the drug from an aerosol canister, preferably a metered dose inhaler.

Conventional aerosol canisters, such as those of aluminum, glass, stainless steel, or polyethylene terephthalate, can be used to contain the medicinal formulations according to the present invention. Aerosol canisters equipped with conventional valves, preferably, metered dose valves, can be used to deliver the formulations of the invention. The selection of the appropriate valve assembly typically depends on the components in the medicinal formulation.

### Cosolvent and Other Additives

Medicinal formulations according to the present invention can include an optional cosolvent or mixtures of cosolvents. The cosolvent can be used in an amount effective to dissolve the drug and/or the biocompatible polymeric compound. Preferably, the cosolvent is used in an amount of about 0.01-25% by weight based on the total weight of the formulation). Non-limiting examples of suitable cosolvents include ethanol, isopropanol, acetone, ethyl lactate, dimethyl ether, menthol, tetrahydrofuran, and ethyl acetate. In one aspect, ethanol is a preferred cosolvent. It may be desirable to choose the cosolvent so as to ensure compatibility with the biocompatible polymeric compound and other formulation ingredients. In some instances isopropanol or a less nucleophilic solvent may be preferred.

Other additives (i.e., excipients), such as lubricants, surfactants, and taste masking ingredients, can also be included in medicinal formulations of the present invention.

### EXAMPLE

### Synthesis of N,N′-ethylenebis(acetyloligolactyl)amide

D,L-lactic acid (8.3 kg, Mashushino Chemical Co.) was place in a reactor and heated at 150° C. and 40 mbar pressure for 5 hours. After this time, the reaction was cooled to approximately 100° C. The vacuum was removed and the reaction was purged with nitrogen. 4.66 kg acetic anhydride (Fisher Scientific) was added and the reaction heated at 120° C. for 5 hours under a nitrogen purge. After this time, excess acetic anhydride and acetic acid were removed by distillation at 40 mbar and 120° C. To the reaction was then added 2.33 kg of 2-methyl-2-propanol (Sigma Aldrich). This was heated 80° C. for 9 hours under a nitrogen purge. Excess 2-methyl-2-propanol was then removed by distillation at 40 mbar and 120° C. The resulting molten, acetylated oligolactic acid was then heated at 160° C. and <10 mbar for four hours to pyrolyze any t-butyl esters. The product was then refined by passing it through a rolled film evaporator (feed rate 1 kg/hr, jacket temperature=180° C., internal condenser temperature=40° C., pressure=0.05 mbar). The product, acetylated oligolacticacid (3.83 kg) was characterized by NMR spectroscopy and shown to have a degree of polymerization of 8.1.

Acetyl oligolactic acid from the previous step was heated in an oven to 100° C., and 255.7 g (0.40 mol) was added to a 1 gallon stainless steel pressure vessel. To this was added 61.2 g (0.38 mol, 0.95 equiv.) of 1,1′-Carbonylbis-1H-Imidazole and a stir bar. The vessel was sealed and charged with 2.5 kg HFA 134a, and the contents stirred for 20 hours. Another pressure vessel was charged with 10.7 g (0.18 mol, 0.90 equiv.) of ethylenediamine, pressurized with HFA 134a vapor and nitrogen, and the HFA 134a solution from the first vessel was transferred to the new vessel using a high-pressure rated tube. The resulting solution was then stirred for 20 hours. The first pressure vessel was charged with 500 mL of 2.0 molal acetic acid (aq), pressurized with HFA 134a vapor and N₂, and the HFA 134a solution from the second vessel was transferred back to the first vessel. The contents of the first vessel were stirred for 1 hour, then allowed to rest for 30 minutes. The HFA 134a phase was then drained back into the second vessel, and the aqueous phase discarded. The HFA 134a solution was extracted in this fashion two more times. Next, the HFA 134a solution was extracted in similar fashion with three 400 mL portions of 50% saturated NaHCO₃/1.25 molal NaCl (aq) solution. The HFA 134a phase was then transferred to a vessel containing 100 g MgSO₄ and stirred for 4 hours to pre-dry the solution. The solution was then filtered into a clean, dry pressure vessel through a high-pressure rated filter housing equipped with a paper filter (Whatman #5). The pre-dried solution was then further dried by circulating over a column containing 3A molecular sieves for 72 hours using a diaphragm pump. The solution was passed through a filter (Whatman #5) and sprayed into a flame dried glass jar under nitrogen purge, and the majority of the solvent was allowed to evaporate. The jar was placed under high vacuum for 24 hours, and the product isolated as a dry white powder (144.71 g, 61.7% yield).

The relative amount of terminal hydroxy end group impurity (i.e., where the acetyl group is replaced by hydrogen) was determined by measuring the size, A, of the resonance peak (δ=4.35-4.45 ppm) in a proton NMR spectrum for the methine hydrogen adjacent to the terminal hydroxy and comparing this to the size, T, of the resonance peak (δ=2.05-2.15 ppm) for the hydrogens present in the terminal acetyl group for the N,N′-ethylenebis(acetyloligolactyl)amide. For purposes of calculation the assumption is made that all of the terminal hydroxy impurity is present in molecules also having a single acetyl end group, thus the number of intended biocompatible polymer molecules is (T/3−A)/2 when the number of hydroxy impurities is A. The resulting relative amount of hydroxy functional end group impurities to every 100 biocompatible polymer molecules was about 0.3.

The relative amount of carboxylic acid end group impurities was determined by comparing the relative number of non-carboxy end groups to the relative number of bridging groups. The relative number of non-carboxy end groups is (T/3+A) where A and T are measured as described above. The relative number of bridging groups is determine by measuring the size, B, of the resonance peak (δ=3.2-3.6 ppm) for the methylene hydrogens in the bridging group. Complete coupling of the acetyl oligolactic acid prepared above will provide 2 end groups for every bridging group, so the number of extra end groups (i.e., the number of carboxylic acid impurities) is (T/3+A)−(B/2). The number of intended biocompatible polymer molecules is ((B/4)−A), again making the same assumption as before that all of the terminal hydroxy impurity is present in molecules also having a single acetyl end group. The resulting number of carboxylic acid functional group impurities to every 100 biocompatible polymer molecules was about 0.4.

The composition thus had a combined —OH end group impurity level of about 0.7 impurities for every 100 biocompatible polymer molecules. One deficiency, however, was that the NMR method used did not distinguish the difference between end groups on molecules having a single impurity from end groups on molecules having more than one impurity. That is, two molecules, each having a single hydroxy terminal group, give an equivalent response to the combination of one molecule having two hydroxy terminal groups and one molecule having two acetyl terminal groups. Likewise, the combination of one molecule having both a hydroxy and carboxylic acid end group along with one bridged molecule having two acetyl terminal groups gives an equivalent response to two molecules having a single hydroxy and carboxylic acid end group, respectively. In each case (i.e., for determining hydroxy and carboxylic acid end group amounts) this may cause the values noted above to be a slight overestimate, but it is expected that the amount of such impurity present was quite small so that the effect would have been minor.

What is claimed is:

1. A medicinal formulation comprising:
drug;
biocompatible polymer of the formula: 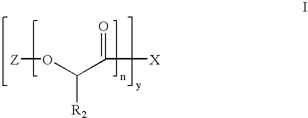
wherein Z is —C(O)R₁;
each R₁ is independently selected from a linear, branched, or cyclic alkyl, alkoxy, or aryl with 1 to 18 carbon atoms optionally substituted by carbonyl, oxy, thio, and/or nitrogen;
each R₂ is independently selected from hydrogen or a linear or branched hydrocarbon with 1 to 4 carbon atoms;
X is selected from the group consisting of —OR₁, —SR₁, —N(R₁)₂ and divalent or trivalent headgroups terminated in oxygen, nitrogen, or sulfur; y is greater than or equal to 1 and less than or equal to 3; and
wherein there is a low level of —OH end group impurity, where Z is —H and/or X is —OH, such that there are no more than 110 of these —OH end groups for every 100 biocompatible polymer molecules.

2. A medicinal formulation according to claim 1, wherein R₁ is alkyl.

3. A medicinal formulation according to claim 2, wherein R₁ is methyl.

4. A medicinal formulation as in claim 1, wherein R₂ is methyl.

5. A medicinal formulation as in claim 1, wherein y is 2.

6. A medicinal formulation according to claim 5 wherein X is —NHCH₂CH₂NH—.

7. A medicinal formulation comprising:
drug;
biocompatible polymer N,N′-ethylenebis(acetyloligolactyl)amide of the formula: 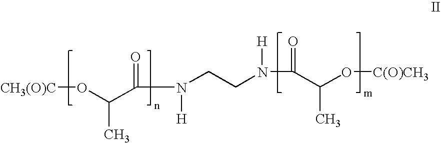
containing at least one unit derived from L-lactic acid and at least one unit derived from D-lactic acid;
wherein the average value of m and n is independently between 6 and 25; and
wherein there is a low level of biocompatible polymer impurities having a hydroxy or carboxylic acid functional end group, such that there are no more than 5 of these —OH end groups for every 100 molecules of the biocompatible polymer N,N′-ethylenebis(acetyloligolactyl)amide.

8. A medicinal formulation as in claim 1, wherein the average value of n is independently between 8 and 11.

9. A medicinal formulation as in claim 1, wherein the average value of n is independently between 11 and 25.

10. A medicinal formulation as in claim 1, further comprising a propellant.

11. A medicinal formulation according to claim 10, wherein the propellant comprises 1,1,1,2-tetrafluoroethane.

12. A medicinal formulation according to claim 10, wherein the propellant comprises 1,1,1,2,3,3,3-heptafluoropropane.

13. A medicinal formulation as in claim 1, wherein the biocompatible polymer comprises units derived from D,L-lactic acid.

14. A medicinal formulation as in claim 1, in which at least half of the stereocenters in the biocompatible polymer are derived from L-lactic acid.

15. A medicinal formulation as in claim 1, in which at least half of the stereocenters in the biocompatible polymer are derived from D-lactic acid.

16. A medicinal formulation as in claim 1, wherein there is a low level of biocompatible polymer impurities having a hydroxy functional end group, such that there is no more than 1 of these —OH end groups for every 100 molecules of the biocompatible polymer.

17. A medicinal formulation as in claim 1, wherein there is a low level of biocompatible polymer impurities having a carboxylic acid functional end group, such that there is no more than 1 of these —OH end groups for every 100 molecules of the biocompatible polymer.

18. A medicinal formulation as in claim 1, wherein there is a low level of biocompatible polymer impurities having a hydroxy or carboxylic acid functional end group, such that there is no more than 1 of these —OH end groups for every 100 molecules of the biocompatible polymer.

19. A medicinal formulation as in claim 1, wherein the polydispersity of the biocompatible polymer is less than or equal to 1.5.

20. A medicinal formulation as in claim 1, wherein the number-average relative molecular mass of the biocompatible polymer is greater than 1200.

21. A medicinal formulation as in claim 1, wherein the ratio of Mn/P of the biocompatible polymer is at least 900.

22. A medicinal formulation as in claim 1, wherein the biocompatible polymer is synthesized by condensation polymerization.

23. A medicinal formulation as in claim 1, wherein the formulation is free of metal-based catalysts.

24. A medicinal formulation as in claim 1, wherein the drug is in solution.

25. A medicinal formulation as in claim 1, wherein the drug is in suspension.

26. A metered dose inhaler including a formulation as in claim 1.

27. A powder comprising a formulation as in claim 1.

28. A dry powder inhaler including a formulation as in claim 1.

29. A medicinal formulation of claim 1, comprising a combination of at least two or more drugs.

30. A medicinal formulation as in claim 29, wherein at least one drug is in suspension and at least one drug is in solution.

31. A method of stabilizing a medicinal formulation in a drug delivery system, the method comprising the steps of preparing a formulation as in claim 1 and utilizing the formulation in a drug delivery system.

32. A method of treating in an animal a condition capable of being treated by a drug, the method comprising the steps of: (i) providing a formulation according to claim 1, and (ii) administering said formulation to said animal.

33. A medicinal formulation comprising:
drug;
biocompatible polymer N,N′-ethylenebis(acetyloligolactyl)amide with an average number of repeat units in each oligolactic acid chain of between 8 and 11;
wherein the polydispersity of the N,N′-ethylenebis(acetyloligolactyl)amide is less than or equal to 1.5; and
wherein there is a low level of biocompatible polymer impurities having a hydroxy or carboxylic acid functional end group, such that there are no more than 5 of these —OH end groups for every 100 molecules of the biocompatible polymer N,N′-ethylenebis(acetyloligolactyl)amide.
